(19) United States
(12) Patent Application Publication
(10) Pub. No.: US 2011/0301617 A1
(43) Pub. Date: Dec. 8, 2011

(54) System and Method for Immobilizing a Tick on a Host

(76) Inventor: Gregory P. Bach, Hamburg, PA (US)

(21) Appl. No.: 13/152,792

(22) Filed: Jun. 3, 2011

Related U.S. Application Data
(60) Provisional application No. 61/352,122, filed on Jun. 7, 2010.

(51) Int. Cl. A61B 17/50; A61P 33/14; A01M 7/00; A61K 31/08

(52) U.S. Cl. 606/131; 514/722; 424/45; 43/132.1

(57) ABSTRACT

Provided is a system and method for immobilizing and killing a tick on a host, allowing for removal of the tick without triggering of the tick's regurgitation reflex. Further provided are compositions and kits related thereto.

### CROSS-REFERENCE TO RELATED APPLICATIONS

The present invention claims priority to U.S. Provisional Application Ser. No. 61/352,122, filed on Jun. 7, 2010, which is incorporated herein in its entirety.

### FIELD OF THE INVENTION

The present invention relates to compositions, articles containing compositions, and systems and methods of use of the compositions, for rapidly and efficaciously immobilizing a tick, which is on or attached to a mammal, permitting its safe and intact removal, thereby preventing, inhibiting or reducing injury or infection of the host by, and the spread of, tick-borne diseases.

### BACKGROUND OF THE INVENTION

The transmission of vector-borne diseases through pests is a problem throughout the world and is best controlled through the control of those vectors. Ticks are arthropods related to spiders, mites and fleas. There are a variety of ticks found throughout the world and they are dependent on animal and human hosts for survival. They are also excellent hosts in turn to many microbial pathogens including bacteria and viruses that are responsible for many diseases of humans and animals. For example, the black legged deer tick (Ixodes scapularis) may transmit Lyme disease to a host in the Eastern and upper mid-Western U.S. If infected, the tick, while feeding on the host's blood, passes an infectious microbe, living in the tick's mid- or hindgut, into the host's bloodstream. On the U.S. West Coast, the black-legged tick (Ixodes pacificus) is involved in the cause of Lyme disease. In Europe and Asia the black-legged ticks that serve as host carriers for Borrelia burgdorferi and cause Lyme disease are Ixodes ricinus and Ixodes persulcatus, respectively.

In the U.S., Lyme disease now accounts for more than 95 percent of all reported tick vector-borne illnesses. In endemic areas 3% or more of the population have been infected by the disease. Between 1982 and 2001 there was a 25 fold increase in the incidence of Lyme disease in humans and the rate of disease progression and geographical area are increasing.

Lyme disease is a multisystem infection caused generally by the bacterium, Borrelia burgdorferi, which is a spirochete that can infect both humans and animals. Lyme disease has a well-established infection pathway involving compatible host reservoirs, such as mice and deer, but may also include mosquitoes. As an essential vector, black-legged ticks transport the infectious pathogen, bacterium and/or spirochete, from mice to humans or other mammalian hosts.

In the U.S. there are other tick-borne diseases of considerable importance to human health. Rocky Mountain Spotted Fever, caused by Rickettsii rickittsi is hosted and transmitted by the tick, Dermacentor variabilis, found in the East and South, while in the West the tick, Dermacentor andersoni, is involved in infection and transmission. A more recently identified disease, Babesiosis, caused by Babesia microti is hosted by Ixodes scapularis and Ixodes pacificus, and transmitted by the bacterium, Babesia equi. This disease and others can be co-transmitted with Lyme disease and resembles malaria since the microbe invades red blood cells.

Another recently identified tick related disease is Erhlichiosis, caused by rickettsial bacteria. There are two distinct forms of the disease. One form known as monocytic ehrlichiosis (HME) is caused by the rickettsial bacterium, Ehrlichia chaffeensis and is hosted and transmitted by the ticks, Dermacentor variabilis and Amblyomma americanum in the South-central and South Atlantic U.S. The second disease form is granulocytic ehrlichiosis (HGE), caused by Ehrlichia equi, hosted and transmitted by the ticks, Ixodes scapularis, Ixodes pacifica and Dermacentor variabilis throughout the U.S. The vectors of this serious disease, associated with some mortality, may also co-transmit Lyme disease. Other hard ticks, including Groundhog tick (Ixodes cookei), American Dog Tick (Dermacentor variabillis), Brown Dog Tick (Rhipicephalus sanguineus), and Lone Star Tick (Amblyomma americanum) are less common carriers of the Borrelia burgdorferi bacterium directly to humans in North America, but are known reservoirs of Lyme disease.

Although more rare, the following diseases are also cause by tick-borne pathogens: Relapsing Fever, found principally in the western U.S., is caused by Borrelia hermsii, is transmitted by the soft belly tick, Ornithodoros hermsii. Colorado Tick Fever (Mountain), also found in the West, is caused by Colt virus that is hosted by the tick, Dermacentor andersoni. Tularemia (Deer Fly Fever or Rabbit Fever) is caused by the microbe, Francisella tularensis. Tick Paralysis, although not related to a microbe, is caused by a tick-produced toxin from Dermacentor andersoni or Dermacentor variabilis ticks, depending on location.

However pest control over the ticks is often difficult to achieve. Pesticides may be applied in the locus of the ticks or directly on the tick to operate as topical or ingestible pest toxins, e.g., U.S. Pat. Nos. 7,230,033 and 7,629,387, but this may cause widespread environmental danger, also killing beneficial insects or worse. Many pesticides are toxic to humans and animals and may pollute the environment. Hence, a number of commonly used pesticides, such as organophosphates, have been restricted or made commercially unavailable. While biopesticides derived from natural sources, such as plants, fungi, or other natural products, may offer a safer alternative to chemically synthesized pesticides, many biopesticides offer substantially weaker control of pests, or control only a limited spectrum of pests. Yet, even some of the biopesticides, such as pyrethrins, have proven to be environmentally toxic. Additionally, pests may become resistant to certain compounds after continued use. For example, insect resistance to pyrethrins already has been observed.

Ticks have life cycles starting with eggs produced by adult females. These hatch and progress to larvae, nymphs, and adults. Blacklegged ticks require at least two years to complete their life cycle. Larvae begin to hatch in June and feed throughout the summer on white-footed mice or other small mammals or ground-foraging birds. They usually spend the winter as nymphs. The following year the nymphs take blood meals in spring or early summer, feeding on small mammals, birds, or humans. They usually turn into adults by late summer. Adults take blood meals in the fall or the following spring. They feed on white-tailed deer, dogs, horses, raccoons, or humans. Females die after laying eggs in late spring, completing the life cycle.

With Lyme disease, the bite of the nymph offers the greatest risks to humans and animals, the bite of the adult is less so. The disease-causing pathogen of Lyme disease, the spirochete, is found in the gut of the black-legged ticks, where it remains inactive until warm blood enters the gut and enables it to grow and then move to the mouth of the tick. This process takes anywhere from 24 to 36 hours. However, other tick-borne disease pathogens have even shorter growth periods. The soft-shelled tick, Ornithodoros hermsi that causes Relapsing Fever, requires only one hour or less to be available for infection at the bite site. See, Hauser, in “Outwitting Ticks,” The Lyons Press, Connecticut (2001). Therefore, embedded ticks of all types should be removed as quickly as possible. In the case of Lyme disease the tick should be removed before 24 hours after the time of attachment. Unfortunately, however, the safe removal process is not always so simple.

Ticks, whether in their larvae, pupa or adult state, adhere to the skin by a rostrum that penetrates the epidermis of the host mammal, such as a human, and permits the tick to become engorged with the host's blood. Attachment is initially achieved by the secretion of cement substances that allow the tick to attach to the host. Traditionally, these ticks are removed by hand, grasping them between the fingers, or with tweezers, forceps or instruments of various designs. Such instruments and devices for physically removing ticks are disclosed, e.g., in U.S. Pat. Nos. 6,206,892; 6,106,041; 5,998,762; 5,876,409; 5,843,094; 5,792,148; 5,607,434; 5,595,569; 5,554,161; 5,447,511; 5,407,243; 5,137,318; 5,116,347; 5,078,729; 5,002,323; 4,979,771; 4,976,718; 4,938,764; 4,442,837; 4,303,268; and 4,213,460.

However, commercially available tick removal devices tend to be complex and relatively expensive, yet most fail to effectively remove a wide variety of the tick species typically encountered by animals and humans, such as the smaller Ixodes tick responsible for Lyme disease. In general, however, success depends on the type of tick, its developmental stage, and depth of attachment. Note that nymphs that penetrate deeply are less likely to be removed intact by any method. Often the available tick-removal methods and devices fail to safely remove the entire tick from the host, and may sever the head from the tick in the vicinity of its dorsal scutum, leaving the head and rostrum embedded in the host's skin. This can result in infection at the site, but more importantly, the remaining head and mouth of an infected tick may still contain 500-1000 spirochetes to infect the host, even after the tick has been partially removed. Grasping the tick may squeeze the tick's posterior, often resulting in injecting disease-causing organisms into the host, and painfully pulling the skin and any of the host's fur or hair caught in the device.

Moreover, when the tick releases from the host, it automatically regurgitates a small portion of blood and saliva, leaving it behind in the flesh of the host. In the case of Lyme disease, medical research has recently concluded that the responsible spirochete is injected into the host during the regurgitation process. As a result, Lyme disease is most often contracted, not because a host was bitten by a tick, but rather because the tick was improperly removed, driving the pathogens, including spirochetes, virus, rickettsiae, bacteria, and protozoa that cause a wide array of infectious diseases, into the host's blood stream. Accordingly, many of the common methods used to remove ticks are improper, and in some cases can actually promote the spread of Lyme and other tick-borne diseases.

Others have designed tick removal devices that add heat and/or light to the physical removal process, such as U.S. Pat. Nos. 6,100,501; 5,556,563; 5,276,306; 4,979,771 and 4,213,460, yet the problems preventing easily and safely removing the entire tick from the host without expressing the disease-bearing pathogens into the host have remained. Further, as the tick backs away from the host in an attempt to escape the burning or fumes, it may activate its regurgitation process and inject disease causing organisms into the host.

Shampoos have been specially formulated to remove ticks, such as those disclosed in U.S. Pat. Nos. 6,683,065 and 4,668,434. However, while safe for human and animal use these methods may not be as effective due to dilution of the effective chemicals, plus they have the disadvantage of not being available for rapid use on the host on site where ticks are found.

Alternative methods have applied chemical compositions to the tick, such as U.S. Pat. No. 6,103,758. However, many of the chemicals can be unhealthy and adversely react with human skin causing irritations, burning or rashes, and the chemicals can also cause the tick to regurgitate as it attempts to escape. Chemicals may also subject the host to discomfort, and the chemicals may irritate the wound, thus requiring subsequent cleaning. In the case of inventions using keratolytic and/or exfoliant agents, such as salicylic or glycolic acid or their analogs, such as U.S. Pat. Nos. 7,604,814 and Publ. Nos. 2009/0270354; 2008/0057018 and 2006/0153935, there is risk of significant pain, tissue damage or scarring of the skin or tissue of the human or animal host.

U.S. Pat. Nos. 4,834,967 or 6,808,717 and Publ. No. 2007/0092545 disclose an aerosol coolant spray for killing and removing ticks comprising a coolant spray for the delivery of an essential oil active agent. In the '967 patent, the use of Freon, dry ice (carbon dioxide) or liquid nitrogen directly onto an object no larger than a poppy seed (the size of a deer tick) attached to the skin of a host would cause significant burning, skin damage and scarring if it were to touch the host's skin. Although the patent indicates the ability to limit the composition only on the pest with no reported pain or discomfort, common usage in the field makes avoiding getting the identified refrigerants onto the host skin an impossibility—particularly for the removal of deer ticks. Therefore, use of the patented method of the '967 patent is simply too dangerous to use on ticks that have attached to the skin of viable humans or animals. U.S. Pat. No. 5,317,041 provides an invention comprising citrus peel oil (d-limonene), a carrier fluid (isopropyl alcohol), and an oil solubilizer (polyoxyethylene (20) sorbitan monooleate). However, the presence of oils or essential oils, such as those used in the '717 or '041 patents or the 2007 Publication have been shown to cause regurgitation by a tick, propelling disease-causing pathogens or spirochetes back into the host's blood stream. The use of aerosols comprising ethyl chloride or commercially-available tick freezing spray on the market under the trade name Tickner (active components are not provided), is distinguished from the present invention, which requires no damaging, wide-area spray delivery.

Accordingly, it is clear that despite the availability of numerous tick removal methods, until the present invention, a long felt need has remained in the art for a composition and method that offers safety, reliability and effectiveness for removing a tick from a host without triggering the regurgitation reflex that delivers any tick-borne pathogen from the tick into the blood of the host, wherein the method and system is safe and easily applied to or by the host, without residual damage to the environment.

### SUMMARY OF THE INVENTION

Ticks are parasitic, living on the blood of a mammalian host, and frequently tick bites deliver or leave behind disease causing microorganisms in the blood of host. Ideally, a tick would not be permitted to contact a mammalian host or to imbed its head and hard exoskeletal mouth parts into the skin and tissue of the host. Once the host's skin is broken, however, it is essential to remove the tick as rapidly as possible from the host, in a way that does not involve human contact with the viable parasite or activate its autonomic reflex process of regurgitation.

It is, therefore, an object of this invention to meet the foregoing need and provide a composition that rapidly or instantaneously immobilizes the tick, and its method of use that occurs so quickly that the tick is immobilized and/or dead before the regurgitation reflex is activated. Accordingly, in accordance with the present invention, the tick is instantaneously immobilized and/or very rapidly or instantaneously killed, before disease-causing pathogens are delivered into the blood stream of the mammalian host. Advantageously, the present safe and effective immobilization system and method prevents, inhibits or reduces the spread of tick-borne disease to or in a host, and permits safe immediate emergency removal of the tick from the host in the field or forest where ticks are found and attach to hosts, until follow-up medical attention may be sought.

It is also an object of the invention to immobilize the tick with minimal dexterity, leaving at least one hand free to maintain the location of the wound for subsequent cleansing, examination and applying medication, and/or to calm a child or animal host.

The invention will be more fully understood from the following detailed description of preferred embodiments, drawings and examples, all of which are intended to be for illustrative purposes only, and not intended in any way to limit the invention.

### DETAILED DESCRIPTION OF CERTAIN EMBODIMENTS OF THE INVENTION

In recent years, there has been an alarming increase in the incidence and geographical range of tick-borne disease. Ticks are geographically distributed throughout most of the United States and, in various species, across many other areas of the world. These various tick species are vectors for the transmission of many serious diseases, including Lyme disease, Rocky Mountain Spotted Fever in the Western Hemisphere and various related rickettsial diseases in the Mediterranean area, Africa, Australia, India, Russia and elsewhere. Both human and animal hosts are affected by a number of infectious agents carried in and/or transmitted by ticks.

By “host” is meant any human or mammalian animal (also referred to as a “subject”) that a tick might attach to, and through its bite, transmit disease-causing organisms into the host's blood. Such hosts encompass all mammals, including humans, and including without limitation, veterinary, farm, research, zoo, captive and wild animals, such as canines, felines, equines including, horses, cattle, sheep, goats, deer, primates and the like. The compositions of the invention are administered and the disclosed methods applied to any host, including human or non-human animal, in an amount effective to inhibit tick biting and/or to cause rapid, or even instantaneous, immobilization and/or killing of the tick, permitting safe and effective removal of the tick or other arthropods from the host. Thus, the compositions are useful as anti-tick agents.

Ticks are parasitic members of a group of relatively large blood sucking insects of the order Acari, including the families Ixodidae, Argasidae and Hippoboscidae. All carry diseases. They are wingless creatures, having a barbed proboscis which extends from the head. The proboscis is inserted into the skin of a warm blooded host for the purpose of extracting the mammal's blood, which the tick ingests until its highly elastic body is engorged with the host's blood, stored in the tick's large sac-like abdomen, or gut. At 400× magnification, the mouth and head-end of the tick resembles a long, straight, blade-like shape, with a row of ten to twelve V-shaped, barb-like teeth on each side, which are also inserted into the host's skin for feeding. Through this hollow proboscis passes the host blood into the tick when it feeds on the host, and some consumed blood mixed with tick digestive secretions are passed back into the host, and with it the disease-causing pathogens are transmitted from the gut of an infected tick to the blood of the host.

When a tick attaches to the host, it not only inserts a portion of its mouthparts into the host to reach the blood supply, it also secretes an adherent cement onto the skin of the host which helps prevent the tick from becoming detached from the host. Viruses, bacteria and other microorganisms or infectious agents on, or in, a tick's body can be transmitted to the host during the tick's feeding cycle. However, several hours may elapse after attachment before infectious agents are transmitted to the host. Consequently, rapid, safe removal of the tick from the host—without triggering the regurgitation response—may prevent or substantially reduce delivery of tick-borne pathogens to the blood of the host, even though the tick may have already embedded into the host's skin. Accordingly, the sooner the tick is removed, the lower the risk of infectious agents being transmitted to the host. Therefore, the best method of protecting the host from diseases carried by the tick is a rapid, safe, and effective removal of the tick from the host.

Ticks are further characterized by a sharply defined line of division between the head and thorax-abdomen, and thus the head of an embedded tick is relatively easily separated from the thorax and abdomen when longitudinal stress is applied. The barbs on the proboscis allow the tick to embed its head through the surface and into the skin of the host, and to hold on tenaciously while ingesting blood through the proboscis. In fact, an embedded tick holds on so tenaciously that if an attempt is made to remove the tick by grasping the exposed abdomen and pulling outward, the thorax and abdomen portions readily separate from the head and leave the head remaining in the skin.

The embedded detached head, remaining in the skin is often the source of irritation, inflammation and infection in the host, even if the tick was not carrying a disease-causing pathogen. Of course, if infected, the spirochetes or other bacterium in the tick's embedded mouthparts remaining in the host's skin continue to be transmitted into the host. It is therefore apparent that embedded ticks must be removed intact or killed or immobilized in such a way as to avoid transmission from the tick into the blood stream of the host, thereby preventing or reducing the possibility of serious infection.

Arthropods, including ticks, transmit diseases to hosts, including, but not limited to, Lyme disease, Rocky Mountain spotted fever, Colorado tick fever, tularemia, relapsing fever, tick paralysis, and ricksettial diseases, as well as anaplasmosis, tick borne-babesiosis, equine and St. Louis tick-borne encephalitis, West Nile Virus encephalitis, Eastern equine encephalitis, Venezuelan equine encephalitis, Western equine encephalitis, Lacrosse encephalitis, Q fever, spirochetosis, toxoplasmosis, Dengue Fever; Yellow Fever, powassan-like virus infection, malaria; ehrlichiosis, Plague, and the like (referred to herein as “tick-borne diseases,” the term is intended to broadly further encompass diseases also delivered to hosts by, or caused by, other arthropods).

Lyme disease is a particular concern, the prevention of which is a goal of this invention if the tick is prevented from breaking through the skin of the host. Or if attachment has not been prevented, methods are provided herein to hygienically, and safely remove the tick, preferably including its mouthparts, before any disease-causing pathogen is released into the host's blood stream. After the tick is immobilized and no longer able to transmit disease, it is suggested that the host obtain medical attention to fully remove, or to confirm complete removal of all residual parts of the tick, including the barbed mouthparts, from the host's skin.

The pathogenic microorganisms carried by arthropods, more specifically by ticks range from bacteria to virus to spirochetes or rickettsiae, as set forth above. By non-limiting example, the Borrelia burgdorferi spirochete causes Lyme disease. Hard ticks, including the black-legged ticks (e.g., Ixodes scapularis, Ixodes pacificus, Ixodes ricinus and Ixodes persulcatus, depending on location) is an essential host/carrier for Borrelia burgdorferi, causing Lyme disease. Groundhog tick (Ixodes cookei), American Dog Tick (Dermacentor variabillis), Brown Dog Tick (Rhipicephalus sanguineus), and Lone Star Tick (Amblyomma americanum) are less common carriers of the Borrelia burgdorferi, but are also know to act as reservoirs for the disease.

Rocky Mountain Spotted Fever is caused by Rickettsii rickittsi; babesiosis is caused by Babesia sp. Erhlichiosis is caused by rickettsial bacteria Ehrlichia chaffeensis or Ehrlichia equi. Relapsing Fever is caused by Borrelia hermsii, while Colorado Tick Fever (Mountain) is caused by a virus hosted by a tick. Tularemia (Deer Fly Fever or Rabbit Fever) is caused by Francisella tularensis. These disease and others can be co-transmitted with Lyme disease.

The variety of diseases transmitted by ticks emphasizes the need for compositions and methods that permit rapid immobilization of the tick, preferably before the host's skin is penetrated (“pierced” or “bitten”), but certainly as soon as possible after the tick is felt or seen. Often the tick is a vector for more than one disease; often including co-transmission of Lyme disease. Therefore, the present invention is intended to provide compositions and methods for the rapid immobilization and safe and effective removal of any tick from the host, with the knowledge that ticks bear a variety of diseases, many of which effect infection, paralysis and/or mortality of the host, and all of which are encompassed herein.

Unless the tick is first immobilized or dead, many standard tick removal methods and techniques trigger an autonomic regurgitation response in the live, embedded tick, causing the pathogens, such as the Lyme disease spirochete, to be forced from the mouth of the tick with blood, back into the host's blood. Thus, attempts to remove the live tick from the host often inadvertently cause the regurgitation response, thereby actually promoting the spread of tick-borne diseases. In fact, it is now known that most sufferers of Lyme disease contracted the illness because the live, embedded tick was improperly removed—such as by physical removal with the fingers or with tweezers or forcep-type devices, by applying heat, by dousing the parasite with a chemical, or by rotating the parasite about its longitudinal axis in an effort to dislodge its imbedded proboscis. Therefore, such prior art and commonly used removal methods must be avoided.

The only way to prevent transmission of disease from the tick to the host is by immobilizing the tick to stop, inhibit or reduce disease transmission, or by the removal of the immobilized tick and the adherent cement, if present. However, a problem inherent in mechanical systems is that care must be exercised to avoid squeezing or crushing the body of the tick during removal because saliva, hemolymph, or gut contents, containing infectious agents, can be regurgitated into the human or animal host. For example, grabbing the tick with one's fingers is undesirable, as this squeezes the tick and injecting the spirochete into the host. Often the embedded head of the tick is severed from its body during physical removal, leaving it in the host and causing the release of the spirochetes contained therein.

Touching the parasite is repulsive to most people, and it can also spread diseases borne on the tick's body. Thus, the process of mechanically removing the tick from the host may itself cause the accidental contact with infected tick body fluids. Infectious agents can enter a break in the skin of either the host or the person removing the tick. Consequently, all direct contact with the live tick should be avoided.

Mechanical removal of a tick can also pull out some of the host's hair, causing additional discomfort and pain. Moreover, when removing a tick by hand, such as from a child or animal, a person usually employs a free hand to hold the host in place. As a result, while attempting to remove the tick with the other hand the location of the wound may be lost in the possibly thick hair or fur of the host. Then it may be difficult to subsequently relocate the wound for purposes of examination and applying medication, or the detached tick may be lost, permitting it to reattach to the host. Considerable dexterity is needed to remove a tick using devices, such as tweezers or forceps, as the device itself further obstructs clear view of the tick.

Applying heat, such as a burning match, is undesirable for a variety of reasons, not the least of which is the safety of the host. In addition to being unreliable, when the tick backs away from the host in efforts to escape burning, the organism once again activates its autonomic reflex process of regurgitation which can inject the spirochete into the host. Certainly pressing burning objects against the skin of the host without harming the host, requires considerable dexterity, and can burn the host and/or the person removing the tick.

Other methods involve applying chemicals having relaxant properties onto the tick, such as Vaseline, kerosene, chloroform, alcohol, gasoline, etc., with the intent of causing the tick to release its attachment to the host and withdraw its proboscis. However, not only are such methods unreliable, and frequently have no effect, the chemicals or fumes activate the autonomic reflex process of regurgitation of the parasite as it tries to back away from the host in efforts to get air. Thus, the purpose of safely preventing discharge of the tick-borne pathogens from the live tick is defeated, although the intact tick may be entirely removed intact. Plus, chemicals can cause known injury and skin irritations to the host, particularly when the skin has already been wounded, requiring thorough cleaning, not to mention likely damage to the environment. Moreover, continued use of some pesticidal chemicals can lead to resistance, eventually making the chemicals ineffective.

In addition, even those chemical agents that do stimulate the tick to detach from the host will not also reliably remove the adherent cement excreted by the tick. It usually remains on the skin of the host after the tick detaches. The adherent cement may carry infectious agents itself or it may leave a wound in the skin of the host. In either case, the risk of infection is increased.

Accordingly, each known method for removal of a live and intact tick fail to address the urgent problem of simply safely, and effectively removing a tick from its host in a manner that prevents transmission of disease-borne tick exudates into the host blood. Often children, dogs, cats and other small animals struggle violently when embedded ticks are removed, particularly by any method causing pain, such as hot objects or chemicals. As a result, each known prior art method requires considerable dexterity to simultaneously remove the tick and maintain the location of the tiny wound for cleansing and treatment, often while holding the host still.

If the tick remains alive, even if the tick is removed intact, the removal methods itself will have likely caused the delivery of any disease causing pathogens to the blood of the host. When the tick retracts its mouthparts from the host during its normal feeding process, even without removal efforts, after engorgement, it autonomically regurgitates a small portion of its blood meal and saliva, leaving a deposit behind in the flesh of the host. In the case of Lyme disease, medical research has recently found that the transmitter of the disease is a spirochete that lives in the tick's gut, and when the proboscis is removed from the host during the tick's autonomic reflex, the regurgitation process injects the disease-causing spirochetes, if present, to be injected into the host. As a result, even seemingly safe methods that cause the live, embedded tick to remove its proboscis and back out of the host, are ineffective because the very process of removing the proboscis triggers the regurgitation response, even if the tick is then removed intact. Any method that triggers the tick's regurgitation reflex is ineffective at preventing tick-borne disease in the host, and all known methods may trigger such a response if the tick is alive. See, e.g., Glen R. Needham, in “Evaluation of Five Popular Methods for Tick Removal,” Pediatrics, 75(6) (June, 1985). The known methods simply do not cause the tick to remove itself from the host reliably, nor do they quickly kill the tick.

The invention is embodied by compositions comprising dimethyl ether and propane. However, the composition may not contain oils, emulsions or salicylic acid. The dimethyl ether/propane composition of the present invention was developed as a means for immobilizing the tick by freezing it on contact before the regurgitation response is triggered, permitting the tick to be safely removed intact, regardless of whether feeding had begun or not. Thus, the present invention is directed to a composition for topical administration, comprising dimethyl ether and propane (collectively the “active agents”) in an effective amount.

The term “effective amount” as used herein means an amount of the active agents that is sufficient to rapidly immobilize the tick, permitting removal from a host subject's skin, hair, etc, reducing the risk of a tick bite, and/or reducing the risk of infection by a tick-borne microbe. For example, an “effective amount” of the composition for this purpose can be measured as the amount sufficient to rapidly immobilize and/or kill a tick, causing it to release from a subject without detaching the head, and/or at least substantially reducing or preventing initiation of the regurgitation response that delivers tick-borne pathogens into the host's blood. Preferably, the immobilization process further kills the tick, thereby preventing it from ever biting again. Thus, in a preferred embodiment, the tick is also killed upon exposure to the present composition and methods.

“Immobilized,” or grammatical variants of the term, assume its common meaning in this invention in that the treated tick is instantaneously frozen or locked into a fixed position, unable to move any part of its body, and the regurgitation reflex is blocked, preventing onset or continued delivery of any tick-borne pathogen to the host after the moment of treatment. Without being bound to any one theory, it appears that the tick is actually fractionated by the freezing/immobilizing process of the present invention. “Rapid” when used with regard to the presently defined treatment, means that a response occurs immediately upon contact of an effective amount of the sprayed composition with the tick, or within a time ranging from instantaneous to 1-5 seconds, or 1-10 seconds, or 1-15 seconds, or even 1-30 seconds, but preferably in a time not greater than 15 seconds of contact of the composition to the tick. “Instantaneous” assumes its normal meaning, i.e., immediately upon contact, without appreciable delay (in measured time, less than a 5 second delay, or preferably less than a 3 second delay, or even less than a 1 second delay).

The preferred aerosol comprises dimethyl ether and propane as active agents in the following proportions: 1) greater than 50% dimethyl ether or grater than 75% dimethyl ether, preferably at least 90% dimethyl ether (such as ≧92%, or ≧94%, or ≧95%, or 95-97%); and 2) the remaining content as propane, for example, 50% propane or less, or 25% propane or less, or preferably 10% propane or less, preferably 2-5% propane (or greater 2% propane, such as ≧2%, or ≧3%, or ≧4%). Neither oil, nor isobutene, is included in the aerosol composition of the present invention.

Diethyl ether may be extracted, for example, from cedar heartwood, and then filtered, and evaporated in accordance with recognized methods. Quality can be ascertained chromatographically. Propane is readily available and can be combined with the dimethyl either. However, the dimethyl and propane combination is commercially available at acceptable levels in compositions designed for wart removal, such as DR. SCHOLL'S® FREEZE AWAY COMMON & PLANTAR WART REMOVER™ and COMPOUND W FREEZE OFF™. However, wart removal preparations, even produced by the same manufacturer, that contain salicylic acid and oils, such as DR. SCHOLL'S® DUAL ACTION FREEZE AWAY WART REMOVER COMMON AND PLANTAR™, are not acceptable in the present invention because of the dangerous keratolytic effects of the salicylic acid and because oils or essential oils and emulsions may trigger the regurgitation reflex before the tick is immobilized and killed.

Compounds designed for use on human skin or on animals are regulated in the United States by state and federal agencies, including the Environmental Protection Agency (EPA) and Food and Drug Administration (FDA). Advantageously, however, the compositions in acceptable combination as used in embodiments of the present invention, including dimethyl ether and propane in combination, are already approved for commercial, non-prescription use to remove warts from the skin of human and animal subjects. See, HISTOFREEZER® PORTABLE CRYOSURGICAL SYSTEM KIT™ (OraSure Technologies, Inc), or DR. SCHOLL'S® FREEZE AWAY COMMON & PLANTAR WART REMOVER™ (Schering-Plough HealthCare Products, Memphis, Tenn.). Notably, the HISTOFREEZER® product also contains 3% isobutene, which is not found in the present tick-immobilization invention. Thus, the defined compositions are safe for use as liquids on the skin of the hosts in accordance with these governmental and regulatory considerations, although in this instance the composition is used for the disclosed rapid immobilization and/or death of the attached or surface tick before delivery of the tick-borne diseases, which was not previously considered for previous liquid dimethyl ether and propane compositions.

All chemical compounds include both the L- and D-stereoisomers, as well as either the L- or D-stereoisomer, unless otherwise specified. However, the composition of the present invention is substantially nontoxic to humans and domesticated animals and even if propellants are used, they have minimal adverse effects on the environment.

Because rapid immobilization is required to prevent the regurgitation response in the tick, the tick must not be disturbed until it can also be instantly immobilized and/or killed. Consequently, effective measures for “administering,” meaning applying, the compositions of the present invention directly to the tick on the clothes, hair or skin of the host, or on the host after it has embedded into the skin, comprise topical application. Any of a variety of art-known methods can be used to administer the compositions to the host. For example, the compositions of the disclosure can be formulated for topical administration (e.g., as a lotion, foam, cream, spray, gel, ointment, or the like). Examples of formulations in the market place include topical lotions, foams, creams, soaps, towelettes, wipes, and the like.

Localized, aerosol delivery or delivery into an absorbent tip or swab that is contacted directly to the tick on the host is preferred because the tick is not disturbed, thereby causing the regurgitation reflex to be avoided. In a preferred embodiment, such application is by pinpoint or pencil-point daubing or by localized pinpoint or pencil-point spraying the administered composition, but acceptable delivery methods of application include atomizing, foaming preparations, immersing, coating, spotting and pouring of the defined composition directly onto the tick or arthropod on the host.

There are several patents that disclose various extension arms that are designed to hold conventional “aerosol spray” cans and canisters and have an actuating mechanism that engages and presses the valve plunger nozzle member installed on the valve stem to releases the pressurized material. See, e.g., U.S. Pat. Nos. 2,270,422; 3,716,195; 5,368,202; 5,518,148; and 6,663,307, which represent possible delivery devices if adapted to the purposes of the present invention. There are also exemplary patents that are directed toward various devices that are designed to hold a conventional pressurized “spray gun” handle and have an actuating mechanism that engages and actuates the trigger of the pressurized spray gun nozzle. The following patents illustrate attached extension arms: U.S. Pat. Nos. 4,023,711 and 5,485,960.

In operation, when the trigger at the proximal end of the extension arm is squeezed or the trigger button pressed, an actuator rod is retracted against the spring force of the existing return spring in the trigger pump spray head. When the activating pressure is removed from the trigger at the proximal end of the extension arm, the actuator rod and the trigger of the pump head are returned to their extended position by the spring force of the existing return spring in the trigger pump spray head. Thus, when a user squeezes or presses the trigger, the liquid in the pump spray head is forced by the trigger of the pump spray head to spray from the nozzle, and additional liquid is drawn upwardly through conducting dip tube to the pump head.

In spray application embodiments of the invention, sprays are applied from spray containers, such as a can, bottle or other container, either by means of known pump devices, or by releasing it from a pressurized container, e.g. a pressurized aerosol spray can. Conventional “aerosol spray” cans and canisters contain materials under pressure and are typically actuated by a user pressing a valve plunger nozzle installed on the valve stem that activates the can's valve and releases the pressurized material. Conventional manual “trigger pump spray” containers contain materials that are not under pressure and are typically provided with a removable trigger pump dispensing unit that is threadedly engaged on the top of the container and includes a manually operated trigger that pumps the contents through a spray nozzle by repeatedly squeezing and releasing the trigger.

Spray containers are well known, and have enjoyed wide commercial success. Spray compositions can take various forms, for example, sprays, mists, foams, fumes or fog, all of which will instantaneously immobilize the tick before the regurgitation response occurs, so long as the tick has not been previously disturbed. Unless delivered from standard non-propellant pump containers, such spray compositions further comprise propellants, foaming agents, etc. Many spray containers are capable of delivering a fine and widely dispersed “mist” of a product, which is acceptable for covering a wide surface area.

However, wide spread delivery of the cryogen is disadvantageous to the present invention, as the products are very cold and substantial delivery of the product to the host's skin could cause injury. Consequently, preferred methods of the present invention are provided by a directed or “pinpoint” or pencil-point spray, providing precise delivery or application of the product into small or restricted areas that are preferably no larger than the tick itself. Preferably the host's surrounding skin is not touched by the pinpoint application, although minimal exposure of the surrounding skin can be tolerated. In response to this requirement, many spray containers have provided an extension tube or “spray straw” which is inserted into a spray nozzle of the spray container. See, e.g., U.S. Publ. Appl. 2005/0261753 and U.S. Pat. No. 6,808,717. The extension tube acts to focus delivery of the product to a narrow or pin-point spray diameter, thus avoiding the widely dispersed mist that would otherwise result. This spray can also be applied to a pin point applicator for daubing the tick, contained within the delivery system, or separate from it, such as a pointed swab or Q-tip.

Preferably, the present compositions in the tick-removal system are delivered by a pressurized container as a pinpoint or pencil-point spray, delivered from a pressurized container, directly or through an absorbent applicator. One embodiment uses the generation of near-critical cryogen for direct, localized spraying for both medical (e.g., dermatology, and as an intraoperative surgical assist tool during any tumor resection) and non-medical applications. Current liquid nitrogen (LN₂) dispensers for dermatology depend upon the spontaneous boiling of liquid N₂ through a pinhole nozzle, producing slow and accurate dispensing of the cryogen. U.S. Publ. Pat. No. 2005/0261753 provides an exemplary means for precisely controlling the freeze margins to minimize contacting the cryogen from touching any skin surrounding the tick, and avoids wide destruction of the host's skin tissue. Such a dispenser provides a battery-powered canister having a coil to generate critical propellant pressures of the cryogen (e.g., >600-700 PSI) through a calibrated valve. In this manner, only critical cryogen is released through a tip mechanism, avoiding vapor lock and/or sputtering, although the addition of a battery powered system is more than would probably be needed in the present system.

Any such commercially available dispensing system is suitable for use in the present invention, so long as fine control of the spray is possible through a pinpoint, needle-point or pencil- point tip to permit the tick to be coated, without extending the fringe of the coolant spray to any more of the host's surrounding skin than is necessary. An accompanying cone or guide of various sizes may accompany the dispenser and the open end placed over the tick, so that in operation, only the tick is coated with the cryogen, while reflected spray gets vented out higher along sides of the cone to keep the vented gas from the host's surrounding tissue. The present invention is intended to encompass the use of any available delivery device of this type.

In an alternative, but preferred embodiment, there is actually no direct spray of a liquid from the container. Instead, the cryogen is delivered from the container of the type described above in the DR. SCHOLL'S® FREEZE AWAY COMMON & PLANTAR WART REMOVER™ in which the active cryogen is moved into an absorbent applicator. Preferably the applicator is a tip or swab, preferably one having a pinpoint, needle-point or pencil point tip. However, a flat applicator as used in the DR. SCHOLL'S® container with a flattened end could be used, because when attached to the host, the tick's body is raised above the surface of the skin. A container of the type used in the commercially available DR. SCHOLL'S® product is herein incorporated by reference as a known type of container that can be used to deliver the composition of the present invention to a tick on a host.

Operationally in the absorbent tip or absorbent delivery surface, the cryogen is moved from the container into and saturating the absorbent tip, which may then be carefully touched to the tick for several seconds, without disturbing it, and with minimized touching of the cryogen to the host's skin. This applicator method offers the greatest control over the delivery of the cryogen to the tick without significantly affecting the host's skin, and with less risk of the possibility of overflow of the composition onto the host's skin that could occur using even direct needle-point or pin-point sprays. Extensions may also be used in the absorbent tip type of delivery devices, and the present invention is intended to encompass the use of any available delivery device of this type.

In such a preferred embodiment of the invention the composition comprises an aerosol using the dimethyl ether and propane active agents as propellants, and also as the cryogen. Representative propellants include, but are not limited to, methane, ethane, propane, isopropane, butane, isobutane, butene, pentane, iospentane, neopentane, pentene, hydrofluorocarbons, chlorofluoroacarbons, dimethyl ether, carbon dioxide and mixtures of the foregoing.

A particular method of application may be selected in accordance with the intended objectives of and circumstances related to a particular use. An acceptable dosage of the disclosed composition is that amount that will cover the tick, with minimal direct contact of the composition to the host, or preferably little or no direct contact of the cryogen to the skin of the host. In other words, delivery should localize administration of the composition to only the tick, with minimal exposure to the host, in particular avoiding coverage of any more of the host's skin than is absolutely necessary. Preferably no skin is touched. It is further recommended that any composition that is delivered to the host's skin be quickly removed. However, with proper delivery, particularly using a locally directed applicator of the types described above that minimizes the cryogen fringes from touching the skin of the host, there is no damage to, or residual action on the host or the environment by the present invention.

Acceptable carriers are provided in certain embodiments of the invention, including, for example, but without limitation, additives, adjuvants, stabilizers, preservatives, antioxidants, extenders, solvents, surfactants, antifoaming agents, or other chemical agents. Such carriers may be used in powdered solid, liquid, gas, or gel form, depending on the embodiment and its intended application. Acceptable adjuvants are those materials that assist or enhance the action of a compound or composition, e.g., surfactants and antifoaming agents. However, any particular material may provide a one or more than one “carrier” function, such as “additives,” or “adjuvants” in alternative embodiments. In some instances, the efficacy of a composition may be increased by adding one or more other components that minimize toxicity to hosts or increase the rapid immobilizing and/or killing effect of the present invention.

Examples of liquid carriers include, but are not limited to: water, saline, buffering compounds, or aromatic hydrocarbons, such as xylene, toluene, alkylnaphthalene, phenylxylylethane, hexane, cyclohexane and so on; halogenated hydrocarbons such as chlorobenzene, dichlorobenzene, dichloroethane, trichloroethane and so on; alcohols such as methanol, ethanol, isopropyl alcohol, butanol, hexanol, ethylene glycol and so on; ethers such as diethyl ether, ethylene glycol dimethyl ether, diethylene glycol monomethyl ether, diethylene glycol monoethyl ether, propylene glycol monomethyl ether, tetrahydrofuran, dioxane and so on, so long as the administration of such liquid carrier with the active agents of the present invention do not cause the regurgitation reflex in the tick before it is immobilized. However, compositions that trigger the regurgitation response before the tick can be removed, such as aliphatic or cycloaliphatic hydrocarbons, such as paraffins or cyclohexane, or gums, resins, rosins or oils are not suitable for use in this invention.

The active ingredients of the disclosed compositions for rapidly immobilizing and/or killing the tick before it can regurgitate any disease-causing pathogen, can be combined in any ratio so long as the composition is delivered at an effective amount to achieve its intended tick-immobilizing purpose. Accordingly, delivery of the tick-borne disease into the host blood is prevented, or the risk of such delivery is significantly inhibited or reduced. While “preventing” the delivery of tick-borne disease is preferred, equal to ˜100% of the time, it would be difficult to know in field usage whether 100% intervention had been achieved. But delivery after the tick has been immobilized can be prevented because the tick is fractionated at hardened by freezing. Therefore because disease transmission could have begun before immobilization, the system can then achieve “reduction” or “reduce” disease transmission, meaning a lowering or lessening of the amount of tick-borne disease cells that are transmitted to the host blood by at least 1%, 10%, 25%, 50%, 75%, 89%, 90% or more, thereby lowering the risk of infection to 90% of that of the host bitten by an untreated or disturbed tick, or those percentages may be reduced to 80%, 70%, 60%, 50% 30% 20%, or down to 10% or less, dropping to as little as ≦1% of the risk of the host otherwise bitten by an untreated or disturbed tick, if the tick is carrying a pathogen. Or treatment reduces the delivery of pathogens as described, as compared to the risk that would otherwise result if the tick remained on the host or if it were left embedded in the skin of the host, or if it were removed by methods that caused regurgitation by the tick, or if the tick were permitted to complete feeding and withdraw on its own, leaving behind residual blood mixed with pathogens or other infectious agents. As used herein, the terms “treating” and “treatment” of the tick, meaning immobilizing the tick by the present system are intended to include the terms “attenuating,” “preventing” and “reducing” tick-borne disease.

“Inhibiting” means lessening or ameliorating a sign or symptoms of a tick-borne infection or bite by reducing exposure of the host to the tick or the tick's regurgitated material, and by rapidly immobilizing the tick and removing it, to prevent the regurgitation response that could deliver additional pathogens, e.g., spirochetes, into the host's blood. “Inhibiting” may not prevent disease in the host, but it significantly reduces the risk of disease (by at least a 50% reduction, or to as much as a 75% or 90% or 99% reduction), by minimizing the host's exposure to the tick, and by immobilizing the tick before attempting to remove it so that additional pathogenic microorganisms are not forced into the host from the tick. “Inhibiting” also permits the host to safely seek medical attention without extended exposure to the tick for the additional time that it make take to get from the field or forest to such medical facility.

The efficacy of a compound or composition for killing the tick or other arthropod is determined from an adverse effect on the tick population, including (but not limited to) inhibition or elimination of pest feeding activity, or instant immobilization and/or death of the tick as defined above, all of which are encompassed by the term “controlling” the tick. Thus, an “effective amount” of a compound or composition is that amount that rapidly kills or instantly immobilizes the tick. The efficacy and quantity needed to do so, may be determined by routine screening procedures employing known methods to evaluate pesticidal activity and efficacy, such as those screening described in the Examples. In any case, the compositions of the present invention when administered directly to the tick by any of the proposed delivery methods, in an effective amount that covers the tick, but preferably does not touch, or only minimally touches the host's skin. This will immediately immobilize and/or rapidly kill the tick, but have minimal or no adverse effect on plants, non-human animals, or humans that may come into contact with the compound or composition.

The compounds and compositions disclosed herein, have a broad range of tick killing and control effects, since ticks and arthropods represent a variety of types and sizes. While some concentrations of, for example, the dimethyl ether and propane composition, may be more effective on some pests than others, depending in part on concentration, any differences in efficacy should not in any way detract from the utility of these compounds or compositions, or their methods of use for their intended purpose, primarily for preventing the spread of Lyme or other tick-borne diseases. In any particular embodiment, the composition is administered in an amount to rapidly effect tick immobilization and/or death before the regurgitation response delivers the tick-borne pathogen to the host blood. That amount may depend on a variety of factors, including but not limited to, the area to be treated, the tick or arthropod being to be treated, its metabolism, its behavior (e.g., feeding habits, breeding, daily or seasonal activity cycles, development, etc.), and behavior of the host.

In certain embodiments, the defined composition is applied once, while alternative embodiments employ plural applications of the same composition, or may include different compounds or compositions. In any particular embodiment, the frequency of application may be regular or irregular, and the time elapsed between successive applications may very close together to achieve rapid immobilizing or killing effect on the treated tick, without pain (although the product is very cold), or permanent harm or injury to the host.

Further provided are embodiments of the invention providing methods for preventing, inhibiting or reducing the risk of a host's infection by a tick-borne pathogen, or a tick-related bacterial or viral associated disease or disorder. The method provides for administering an effective amount of a composition of the disclosure, comprising dimethyl and propane, directly onto a tick on a host subject who is at risk of being bitten by the tick, or to the host into which the tick has embedded its mouthparts. The term “preventing” means blocking the future delivery of a tick-borne pathogen into a host from that tick by safely immobilizing and/or removing the tick on the host clothing, hair or skin, preferably by rapidly killing and removing the tick before the host's skin is broken. However, such preventing measure cannot prevent the delivery of tick-borne pathogens already injected into the host before treatment of the tick.

Preventing delivery of a tick-borne pathogen into a host by the removal of the tick after the tick has embedded its mouthparts into the skin of the host is much more difficult, but the methods of the present invention may also block entry of the pathogens into the host's blood if the tick is rapidly immobilized, preventing the regurgitation reflex, and removal of the dead tick. However, medical attention is proposed as removal of the tick is, however, only a first step. The wound must be properly washed and treated, followed by recommended examination by qualified personnel. Advantageously, the rapid immobilization of the tick, made possible by the compositions and methods of the present invention, permits the immediate, emergency removal of the disease-bearing tick, even while the host is still in the fields or woods that are frequently inhabited by such ticks. The disclosed treatments do not require special conditions or medical facilities to permit the rapid immobilization and removal of the tick, but the methods do permit time for the bitten host to get to a medical facility for proper treatment of the wound, and to obtain disease prevention measures or treatment by qualified medical personnel.

If the tick has embedded into the host's skin, ingested blood and saliva may have been released into the host during the feeding process and transmission of the disease may no longer be preventable, but once the tick is seen, it can be removed in accordance with the disclosed method to inhibit the spread of tick-borne diseases or the risk thereof is reduced. Therefore, as used in the present invention, “inhibiting” or “reducing” is a best-case alternative to “preventing” tick-borne disease transmission.

The method of the invention may in one or more embodiments be made more effective, not only for instantly immobilizing and/or killing the tick to effect its removal, and to prevent, inhibit or reduce the spread of tick-borne disease, but certain additional components may be applied at the time of treatment, with or in sequence with the applied composition that will facilitate treatment of the host. For example, at least one astringent may be applied to the host (between about 1% w/v to about 10% w/v) to constrict blood vessels of the host in the vicinity of the embedded tick to reduce transport to the host's blood stream of any tick-borne pathogens that may be released until the tick is immobilized and removed. The astringent may be, for example, Witch Hazel or aluminum salts, including, but not limited to, aluminum sulfate, aluminum phosphate and aluminum acetate.

In another embodiment, the compositions or methods of the invention further comprise an effective amount of at least one anti-infective agent applied to the host (between about 1% w/v to about 10% w/v). Such agents include, but are not limited to: benzalkonium chloride, menthol, neomycin, bacitracin and polymyxin. Thus, the anti-tick compositions may include antiviral agents, and/or antibacterial agents. Other therapeutic agents, and/or antibiotic(s) if medically approved, can be administered, simultaneously or sequentially. Suitable antibiotics include, but are not limited to doxycyclines, aminoglycosides (e.g., gentamicin), beta-lactams (e.g., penicillins and cephalosporins), quinolones (e.g., ciprofloxacin), and novobiocin.

Generally, the antibiotic is administered in a bactericidal amount. However, the embodied method provides for increasing antibiotic activity by permeabilizing the bacterial outer membrane, meaning that otherwise sub-inhibitory amounts (an amount lower than the bactericidal amount) of antibiotic can be administered. A “bactericidal amount” is an amount sufficient in the blood to achieve a bacteria-killing concentration, or a bacterial-killing local concentration in the subject receiving the treatment. In accordance with its conventional definition, an “antibiotic,” as used herein, is a chemical substance that, in dilute solutions, inhibits the growth of, or kills microorganisms. Also encompassed by this term are synthetic antibiotics (e.g., analogs) known in the art.

Of course, to be effective, the present invention is advantageously provided in a small, easily carried container, such as a small, aerosol container that can be readily carried in a pocket or backpack so that it may be used as soon as possible, preferably within hours, after the tick is felt, seen or attaches to the host. It is often not possible to bring a person or animal to a medical facility with a supply of chemical agents quickly enough. Therefore, for emergency removal of the tick, kits and supplies are provided for indoor or outdoor use on humans or animals. Alternatively, containers, such as delivery devices described above, may be provided at outdoor stations, such as at tees on a golf course, to permit ticks to be safely, rapidly and easily removed from a host until medical attention can be provided. Moreover, because of the safety and effectiveness of the disclosed compositions and methods for immobilizing and/or killing and removing a tick, particularly an embedded tick, without causing the release of tick-borne pathogens, the disclosed invention is advantageously combined with medical treatment, even at hospitals.

The invention also provides kits or article of manufacture comprising a composition of the invention in a delivery device or vessel, such as a tube, barrel, bottle, bottle, or can, and a written documentation associated the kit or article. The article of manufacture or kit may be, for example, but not limited to an aerosol or spray container, bottle or any other means for storing an aqueous solution or aerosol, either in pump device or aerosol container, or in a container of a ready-to-use formulation for personal, household or field use, from which liquid can be administered, sprayed, foamed, fogged, misted, brushed, dabbed, spotted or poured directly onto the tick or arthropod on the host. The article may be any shape, such as cylindrical, spherical, cubic or conic and any size. The article may be any material suitable for storing the herein described composition, such as any plastics, metals or glass, or combinations thereof, with different parts comprising different materials in the invention.

Advantageously, provided with the kit is a cleaning solution, for cleansing the wound area on the host. Such cleansers are readily supplied in individual towelettes, as are commercially available in individual, single use, disposable packages. Also provided in the kit may be a small tape or envelope, bag or container for securing the removed tick if the host wishes to retain it for further medical examination.

Writing associated with the article or kit indicates that the composition is useful for rapidly immobilizing and/or killing a tick, and for safely removing the treated tick from the host. The writing may be, for example, but not limited to, listing of ingredients, and/or information regarding tick-borne diseases, and/or instructions for using the herein described composition and method to safely remove ticks from a person or animals skin. The instructions may be attached directly to the article, or packaged separately with the article. Labels may indicate the composition is approved for use as a direct-contact pesticide for rapidly killing ticks, and the instructions may specify the pests intended to be controlled by the composition, the method and rate of application, dilution protocols, use precautions, and the like.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art. Terms used herein are intended to be read in terms of conventional usage in the art, and chemical terms are exemplified by The Grant Hackh's Chemical Dictionary (1987).

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

I claim:

1. A method of immobilizing a tick on a host, comprising administering directly to the tick a composition comprising dimethyl ether and propane in an amount sufficient to effect rapidly immobilizing the tick on the host, without triggering regurgitation by the tick.

2. The method of claim 1, wherein the immobilized tick has not pierced the skin of the host, and further removing the tick from the host.

3. The method of claim 1, wherein the immobilized tick has embedded into the host, the method further comprising causing the tick to disengage from the host, permitting removing the tick from the host, and removing same.

4. The method of claim 3, wherein the immobilized tick carries a tick-borne disease.

5. The method of claim 4, wherein the tick-borne disease is Lyme disease, or pathogens co-transmitted with Lyme disease, or both.

6. The method of claim 4, wherein the tick is a black-legged tick from the species Ixodes, American Dog Tick (Dermacentor variabillis), Brown Dog Tick (Rhipicephalus sanguineus), or Lone Star Tick (Amblyomma americanum.

7. The method of claim 6, wherein the tick is a black-legged tick selected from the group consisting of Ixodes scapulari,. Ixodes pacificus, Ixodes ricinus and Ixodes persulcatus, depending on location.

8. The method of claim 4, wherein the step of rapidly immobilizing the tick reduces or prevents transmission of the tick-borne disease to the host.

9. The method of claim 1, further comprising killing the tick.

10. The method of claim 1, wherein the host is human or non-human animal.

11. The method of claim 1, wherein the composition is topically administered to the tick by direct pinpoint or pencil point spraying or by daubing with a flat absorbent applicator containing the composition or with pointed swab or absorbent tip applicator.

12. The method of claim 1, wherein the immobilizing step is instantaneous upon administering the composition to the tick.

13. The composition of method 1, comprising 90% to 98% total volume dimethyl ether, and the remainder of the composition comprising propane, in pressurized liquid form.

14. A method of controlling the spread of tick-borne disease, comprising the method of claim 1.

15. A method of reducing the risk of tick injury to a host, comprising the method of claim 2.

16. A system for immobilizing a tick on a host, comprising a composition comprising dimethyl ether and propane in an amount sufficient to effect rapidly immobilizing the tick on the host, without triggering regurgitation by the tick.

17. The system of claim 16, wherein the immobilized tick has not pierced the skin of the host, and further removing the tick from the host.

18. The system of claim 16, wherein the immobilized tick has embedded into the host, the method further comprising causing the tick to disengage from the host, permitting removing the tick from the host, and removing same.

19. The system of claim 18, wherein the immobilized tick carries a tick-borne disease.

20. The system of claim 19, wherein the tick-borne disease is Lyme disease, or pathogens co-transmitted with Lyme disease, or both.

21. The system of claim 19, wherein the tick is a black-legged tick from the species Ixodes, American Dog Tick (Dermacentor variabillis), Brown Dog Tick (Rhipicephalus sanguineus), or Lone Star Tick (Amblyomma americanum.

22. The system of claim 21, wherein the tick is a black-legged tick selected from the group consisting of Ixodes scapulari, Ixodes pacificus, Ixodes ricinus and Ixodes persulcatus, depending on location The system of claim 19 wherein the tick is Ixodes scapularis.

23. The system of claim 19, wherein the step of rapidly immobilizing the tick reduces or prevents transmission of the tick-borne disease to the host.

24. The system of claim 16, further comprising killing the tick.

25. The system of claim 15, wherein the host is human or non-human animal.

26. The system of claim 16, wherein the composition is topically administered to the tick by direct pinpoint or pencil point spraying or by daubing with a flat absorbent applicator containing the composition or with pointed swab or absorbent tip applicator.

27. The system of claim 16, wherein the immobilizing step is instantaneous upon administering the composition to the tick.

28. The composition of system 16, comprising 90% to 98% total volume dimethyl ether, and the remainder of the composition comprising propane, in pressurized liquid form

29. A kit comprising the system of claim 16 in a portable container, and a set of instructions for use of the system, with associated information.

30. The kit of claim 28, further comprising cleansing material, and tape, envelope, bag or container for holding the removed tick.
